(19) United States
(12) Patent Application Publication
(10) Pub. No.: US 2024/0252379 A1
(43) Pub. Date: Aug. 1, 2024

(54) ORTHOSIS SYSTEM WITH INTERCHANGEABLE EFFECTOR ASSEMBLY

(71) Applicant: Neurolutions, Inc., Santa Cruz, CA (US)

(72) Inventors: Kern Bhugra, Santa Cruz, CA (US); August Anderson, Watsonville, CA (US); Eric Claude Leuthardt, St. Louis, MO (US)

(73) Assignee: Neurolutions, Inc., Santa Cruz, CA (US)

(21) Appl. No.: 18/631,662

(22) Filed: Apr. 10, 2024

Related U.S. Application Data
(63) Continuation of application No. PCT/IB22/59370, filed on Sep. 30, 2022.
(60) Provisional application No. 63/262,471, filed on Oct. 13, 2021.

(51) Int. Cl. A61H 1/02; A61F 2/58; A61F 2/72; B25J 15/12; G06F 3/01

(52) U.S. Cl. CPC A61H 1/0288; A61F 2/588; A61F 2/72; B25J 15/12; A61H 2201/1207; A61H 2201/14; A61H 2201/165; A61H 2201/1685; A61H 2201/5074; A61H 2230/105; G06F 3/015

(57) ABSTRACT

Orthosis systems are disclosed, having a wearable assembly and a user replaceable orthosis effector assembly. The wearable assembly includes a motor mechanism and a coupler that is movable by the motor mechanism between a starting location and an unloading location. The coupler has a receiving space. The effector assembly couples to the wearable assembly and has a tension element that actuates the effector assembly. A first end of the tension element is seated in the receiving space of the coupler when the effector assembly is coupled to the wearable assembly. The first end of the tension element is releasable from the receiving space when the coupler is in the unloading location, decoupling the effector assembly from the wearable assembly.

### RELATED APPLICATIONS

This application is a continuation of International Application No. PCT/IB2022/059370, filed Sep. 30, 2022, and entitled “Orthosis System with Interchangeable Effector Assembly”; which claims priority to U.S. Provisional Patent Application No. 63/262,471, filed Oct. 13, 2021, and entitled “Orthosis System with Interchangeable Effector Assembly”; all of which are incorporated by reference herein in their entirety for all purposes.

### BACKGROUND

Orthosis devices are external apparatuses that help improve the function or alignment of spinal or limb disorders. Orthoses can be utilized to move or assist in the movement of a subject's body part, for example, upper or lower extremities of a human body. Orthosis device designs are often used in rehabilitating an impaired body part, such as due to damage caused by a stroke event.

Orthoses have been designed with various mechanisms to achieve or assist in the movement of impaired body parts. Some designs involve physically attaching an active movable portion of the orthosis device to the body part that is to be rehabilitated. The active movable portion may then be actuated by a motor or some other motion source, thereby causing movement of the impaired body part secured thereto. Another such mechanism to accomplish or assist in the movement of a body part is through a technique called Functional Electrical Stimulation (FES), which involves the application of mild electrical stimuli to muscles that help the muscles move or improve movement.

Brain-Computer Interface (BCI) technology can be used in conjunction with certain orthosis devices. BCI involves the acquisition and interpretation of brain signals to determine intentions of the person that produced the brain signals and using the determined intentions to carry out intended tasks. BCI technology has been explored in connection with the rehabilitation of impaired body parts, such as arm and hand function that have been affected due to a stroke event, physical injury, or degeneration.

### SUMMARY

In some embodiments, an orthosis system includes a wearable assembly and a user replaceable orthosis effector assembly. The wearable assembly has a motor mechanism and a coupler that is movable by the motor mechanism between a starting location and an unloading location, where the coupler has a receiving space. The user replaceable orthosis effector assembly couples to the wearable assembly. The effector assembly has a tension element that actuates the effector assembly. A first end of the tension element is seated in the receiving space of the coupler when the effector assembly is coupled to the wearable assembly. The first end of the tension element is releasable from the receiving space when the coupler is in the unloading location, decoupling the effector assembly from the wearable assembly.

In some embodiments, a user replaceable orthosis effector assembly couples to a wearable assembly, where the effector assembly includes an effector portion, a tension element, and a securing feature. The tension element moves within the effector portion to actuate the effector portion. The tension element has a first end extending out of an end surface of the effector assembly and a second end attached within the effector portion. The securing feature is at the first end of the tension element.

### BRIEF DESCRIPTION OF THE DRAWINGS

FIGS. 1A and 1B are side view diagrams of an orthosis device for a hand, as known in the art.

FIGS. 2A-2B are block diagrams illustrating a rehabilitation system, in accordance with some embodiments.

FIGS. 3A-3D are isometric views of an orthosis system, in accordance with some embodiments.

FIGS. 4A-4C are views of mechanisms for locking a wearable assembly and a user replaceable orthosis effector assembly together, in accordance with some embodiments.

FIGS. 5A-5B are isometric views of internal components of an orthosis system, in accordance with some embodiments.

FIG. 6 is a partial cross-sectional view of an effector assembly in an orthosis system, in accordance with some embodiments.

FIGS. 7A-7C are partial cross-sectional views of a coupling mechanism of an orthosis system, in accordance with some embodiments.

FIG. 8 is a top isometric view of an effector assembly separated from a wearable assembly, in accordance with some embodiments.

FIG. 9 is a side isometric view of components of FIG. 8, in accordance with some embodiments.

FIGS. 10A-10C are isometric views of an effector assembly being mounted onto an orthosis system, in accordance with some embodiments.

### DETAILED DESCRIPTION

Orthosis systems are disclosed that enable multi-functional usage. The orthosis systems include a wearable base portion to which effector assemblies can be attached to perform tasks or movements by the user. The effector portion of the orthosis system, which provides function-performing capability, is designed to be easily interchanged with other effectors, allowing a user to perform different tasks or rehabilitation exercises with the same base device. The base device, which shall be referred to as a wearable assembly, is worn by the user such as on an arm or leg. The interchanging of effector assemblies can be performed by one-handed operation of the user, which is extremely beneficial for stroke patients who may only have the use of one limb due to impairment of the other limb. Furthermore, having an effector that can be easily replaced by other effectors on the same base wearable assembly beneficially enables patients to perform a variety of exercises or tasks at their home. This multi-functionality of a portable orthosis device in a home setting can improve compliance to exercise regimens, enable new tasks to be performed by the patient, and increase rehabilitation rates.

Embodiments of interchangeable orthosis systems of the present disclosure advantageously utilize an actuation component of an orthosis device not only for functional movement of the device but also to serve as a decoupling and attachment mechanism. In particular, embodiments utilize a tension element of the effector assembly both as an actuator and a coupling component. The tension element can be, for example, a push-pull wire that is within the effector assembly and extends out of the effector assembly to be received by the wearable assembly. By using an actuation component for dual purposes, the number of components that need to be incorporated into the system to provide the interchanging capability is limited, thus limiting costs and complexity.

The term “effector assembly” in this disclosure shall refer to a movable portion of an orthosis device that performs a task, function, or operation for or by a user. Embodiments shall be described primarily for effector assemblies that are for hand and finger usage. However, embodiments can also be applied to end effectors for rehabilitation of other body parts of the upper and lower extremities, such as the arm, shoulder, elbow, wrist, hand, leg, knee, ankle, or foot. Motions performed by the effector assemblies can be controlled by a BCI-based apparatus, where a BCI component of the BCI apparatus can operate using one or more types of signals from the subject such as brain signals, muscle signals, or kinetic signals.

Examples of BCI-based systems for rehabilitating impaired body parts that may be used with embodiments of the present disclosure include devices described in U.S. patent application Ser. No. 17/068,426 ('426 application), which is commonly assigned with the present patent application, and which is incorporated herein by reference. The '426 application describes wearable orthosis device designs that operate to move or assist in the movement of impaired body parts, such as those impaired due to a stroke event, among other conditions described in the '426 application. The '426 application describes an orthosis system that can be operated in one or more of: (i) a BCI mode to move or assist in the movement of the impaired body part based on an intention of the subject determined from an analysis of the brain signals, (ii) a continuous passive mode in which the orthosis system operates to move the impaired body part, and (iii) a volitional mode in which the orthosis system first allows the subject to move or attempt to move the impaired body part in a predefined motion and then operates to move or assist in the predefined motion, such as if the system detects that the impaired body part has not completed the predefined motion.

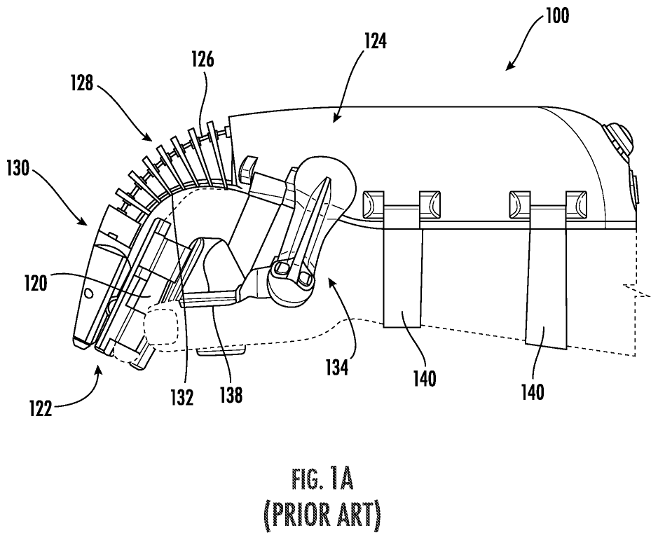

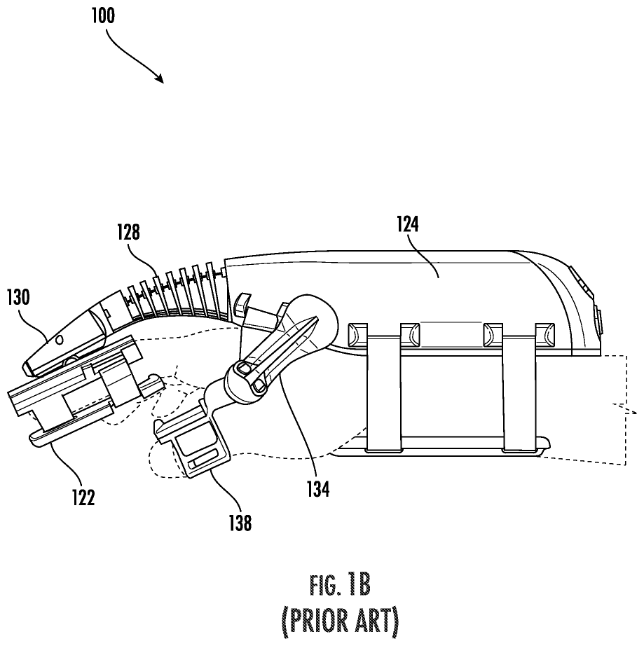

An embodiment of an orthosis device 100 of the '426 application is shown in FIGS. 1A and 1B, where FIG. 1A shows the device 100 in a flexed position while FIG. 1B shows the device 100 in an extended position. The wearable orthosis device 100 may receive transmitted signals (for example, wirelessly) containing information about the brain signals acquired by a brain signal acquisition system (e.g., an EEG-based or electrocorticography-based electrodes headset). The orthosis device 100 may then process those received signals to determine intentions using embedded processing equipment, and in accordance with certain detected patient intentions cause or assist the movement of the patient's hand and/or fingers by robotic or motor-drive actuation of the orthosis device 100.

Orthosis device 100 includes a main housing component 124 configured to be worn on an upper extremity of the subject. The main housing component 124 accommodates straps 140 to removably secure the main housing component 124 and thus the other attached components of the device 100 to the user's body, such as the forearm and top of the hand in this example. The straps 140 may be secured by hook-and-loop, buckle, or other types of fasteners, or may be an elastic band without any fastener required. Each of the straps 140 connects on a bottom of one lateral side of the main housing component 124 and extends around the arm (or leg, in other embodiments) to a bottom of the opposite lateral side of the main housing component 124.

The main housing assembly 124 comprises a motor mechanism configured to actuate movement of a body part of the subject, such as of the upper extremity or lower extremity. A flexible intermediate member 128 is configured to flex or extend responsive to actuation by the motor mechanism to cause the orthosis device 100 to flex or extend the secured body part. In this embodiment, the wearable orthosis device 100 is designed and adapted to assist in the movement of the patient's fingers, specifically the index finger 120 and the adjacent middle finger (not visible in this view), both of which are securely attached to the orthosis device 100 by a finger stay component 122. The patient's thumb is inserted into thumb stay assembly 134 which includes thumb interface component 138. The main housing component 124 is designed and configured to be worn on top of, and against, an upper surface (the dorsal side) of the patient's forearm and hand in this embodiment.

The orthosis device 100 in FIG. 1A is shown in the flexed or closed position. A linear motor device inside the main housing component 124 longitudinally advances and retracts a pushing-and-pulling wire 126 that extends distally from the distal end of the main housing component 124, extends longitudinally through the flexible intermediate structure 128 and connects to a connection point on a force sensing module (FSM) 130. The flexible intermediate component 128 has a flexible baffle structure. When the linear motor in the main housing component 124 pulls the wire 126 proximally, the attached FSM 130 is pulled proximally. The pulling causes the flexible intermediate structure 128 to extend so its distal end is directed more upwardly, causing or assisting in extension movement of the secured index and adjacent middle fingers. The upward movement of the flexible intermediate structure 128 so that its distal end is directed more upwardly (and also its return) is enabled by the baffle structure of the flexible intermediate structure 128. In particular, a generally flat bottom structure 132 is provided on the flexible intermediate structure 128, where the bottom structure 132 is configured to attach to a bottom or hand-side of each of the individual baffle members. The opposite or top-sides of each of the individual baffle members are not so constrained and thus are free to be compressed closer together or expanded further apart by operation of the pushing-and-pulling wire 126 enlarging and/or reducing the top-side distance between the distal end of the main housing component 124 and the proximal end of the FSM 130.

The FSM 130 serves a force sensing purpose, comprising force sensors that are capable of measuring forces caused by patient-induced finger flexion and extension vis-à-vis motor activated movements of the orthosis device 100. The force sensing function of the FSM 130 may be used, for example, to ascertain the degree of flexion and extension ability the patient has without assistance from the orthosis device 100, to determine the degree of motor-activated assistance needed or desired to cause flexion and extension of the fingers during an exercise, or other purposes.

The electro-mechanical orthosis device 100 can be used to acquire significant amounts of meaningful data about a patient's clinical performance, including monitoring utilization data, open/close success rates, force profile characteristics, accelerometer info as well as motor position metrics related to range of motion. For example, force sensors in device 100 (e.g., within FSM 130) can measure passive hand opening force (spasticity), active grip strength and extension force. A six-axis inertial measurement unit (IMU) within main housing component 124, having an accelerometer and gyroscope, is able to monitor motion sensing, orientation, gestures, free-fall, and activity/inactivity. A motor potentiometer within FSM 130, to measure position, facilitates evaluation of the range of motion. The orthosis device 100 has substantial sensor and mechanical capabilities to physically interact with the limb and hand of a stroke patient which can be leveraged to provide various functional metrics.

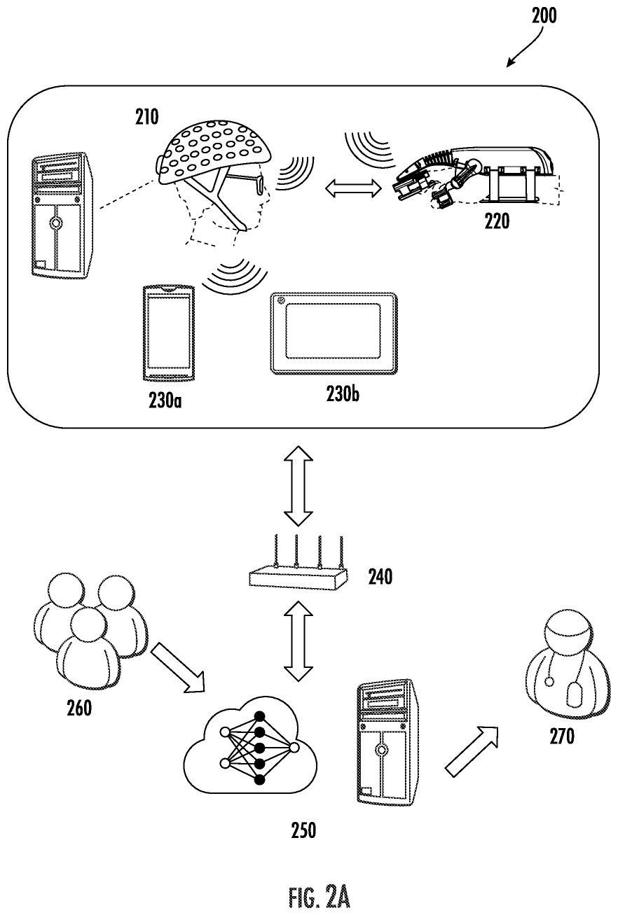

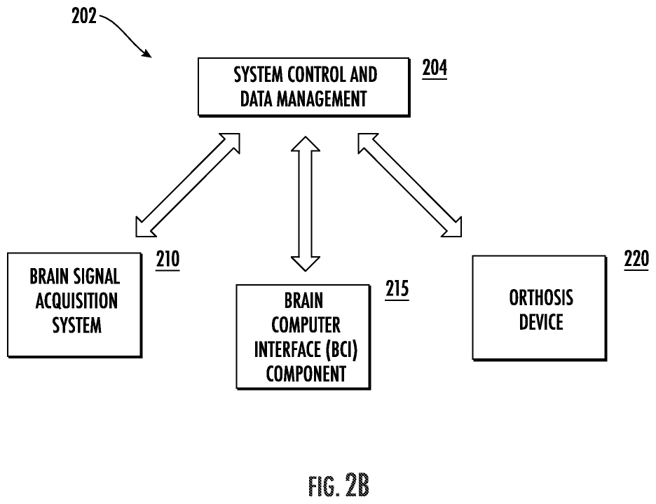

FIG. 2A is a schematic diagram of a system 200 for stroke rehabilitation of a subject, in accordance with some embodiments. System 200 is a home-based brain-controlled interface (BCI) system. System 200 includes a portable brain signal acquisition system 210 (illustrated as a headset) that acquires brain signals from a subject, an orthosis device 220, and a control system 205 configured to operate the orthosis device 220. A computing device such as a smartphone 230a or tablet computer 230b may also be included in system 200 to enable input of information from the subject, to display results for the subject to view, and provide feedback. Smartphone 230a and tablet computer 230b thus also serve as user interface devices. The brain signal acquisition system 210, orthosis device 220, and computing devices (230a or 230b) are all in communication with each other, such as wirelessly. For example, an application program provided on the computing device 230a,b may communicate with the brain signal acquisition system 210 through wireless communications using a protocol such as Bluetooth, and with the orthosis device 220 through wireless communications using a protocol such as Wifi Direct. In some embodiments, the control system 205 may be part of the smartphone 230a and tablet computer 230b, or may be provided as a separate computer server that is in communication with the computing devices 230a,b.

FIG. 2B shows a generalized block diagram of a rehabilitation system 202 using a BCI-based orthosis device such as the device 100 of FIGS. 1A-1B or the device 220 of FIG. 2A. The rehabilitation system 202 may be used for movement of various body parts, for example, an arm, shoulder, elbow, wrist, hand, finger, leg, knee, ankle, foot, or toe. As shown in FIG. 2B, the rehabilitation system 202 includes: (i) a system control and data management component or components 204; (ii) the brain signal acquisition system 210; (iii) a brain computer interface (BCI) component 215; and (iv) the orthosis device 220. The orthosis device 220 may be a body-worn, and thus a portable, body part movement control and/or movement assistance system. The system control and data management system 204 may include not only local control and data management of the system 202, namely, at a site co-located with a subject performing a BCI session (and perhaps integrated with the BCI component 215 and/or the orthosis device 220 or integrated in a local computing device such as a tablet computer), but also may include a remote, network accessible central rehabilitation management computing system. A central rehabilitation management computing system may be used, for example, in set-up and on-going operation of the system, and may be located at a location that is remote of the patient, for example, at a healthcare facility or the facilities of some other type of services provider.

The BCI component 215—which can be provided in the wearable orthosis device 220, computing device (e.g., smartphone 230a or tablet computer 230b), or in headset 210—receives the brain signals from the brain signal acquisition headset 210 and is capable of controlling of a body part interface of the orthosis device 220. The BCI component processes the brain signals to determine the patient's intentions. Control system 205 may be configured to operate the orthosis device 220 in one or more modes, such as the BCI mode, continuous passive mode, and volitional mode as described in the '426 application.

Returning to FIG. 2A, a router 240 sends data from home-based system 200 to a computer processor 250, such as through wireless communications using a WiFi protocol. Computer processor 250 may be a cloud-based system and includes algorithms to process input data from home-based system 200. In some embodiments, data from a larger stroke population database 260 can also be processed for comparison to the patient's status and progress. The computer processor 250 outputs rehabilitation progress information which is then sent back to the patient via router 240 and to a care team 270 which may be the patient's physician, medical care team or other third party.

Although embodiments of orthosis systems in this disclosure shall be described for use with BCI-based systems, the orthosis systems also apply to non-BCI systems. For example, the orthosis systems may be robotic orthosis devices that are manually operated through controls incorporated into the device itself, such as push buttons or touch screen controls in the wearable assembly, or instructions controlled by the user via a smartphone or computer tablet. The manually operated control can provide instructions directly to the orthosis system without the use of brain signals gathered from the patient.

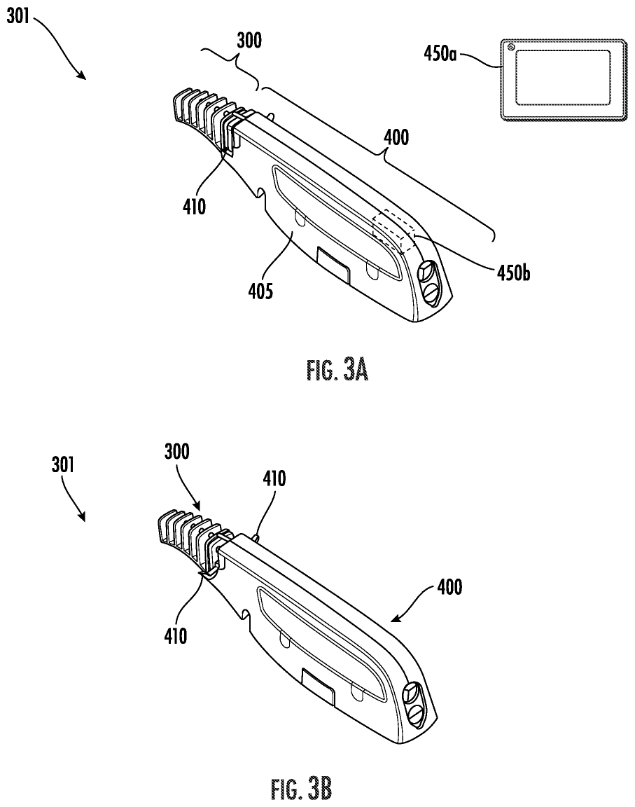

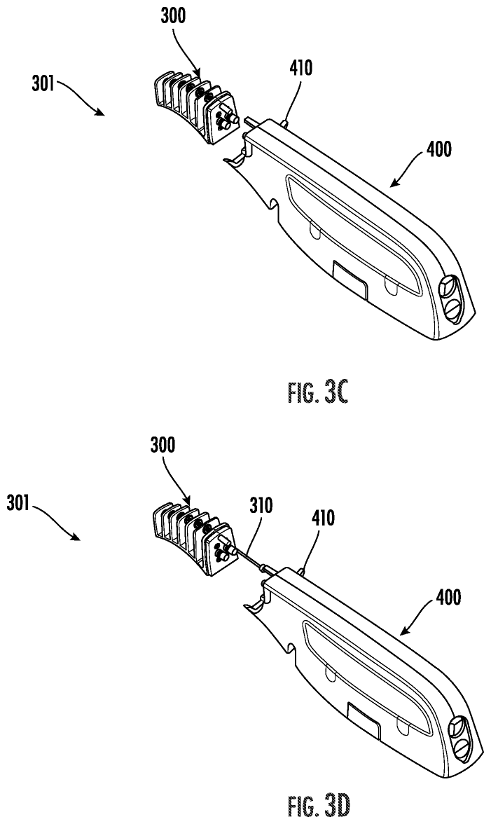

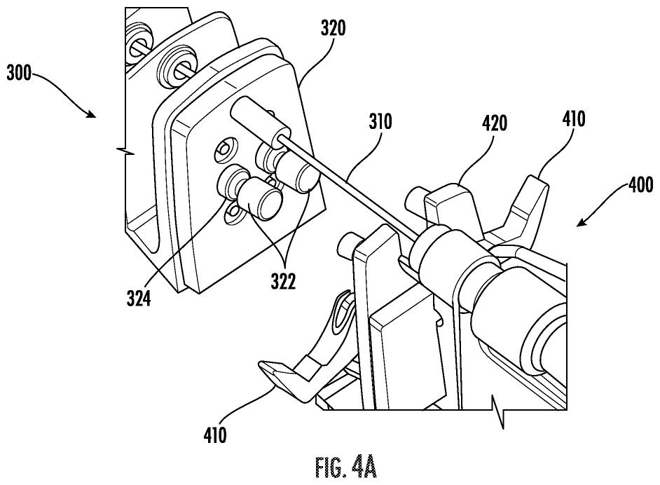

FIGS. 3A-3D are isometric views of an orthosis system 301, in accordance with some embodiments. System 301 includes a user replaceable orthosis effector assembly 300 that couples to a wearable assembly 400. Effector assembly 300 is illustrated similar to the flexible intermediate structure 128 of FIGS. 1A-1B, where some components are omitted from the illustrations for simplicity (e.g., straps, finger stay, thumb stay, force sensing module). In other embodiments, the effector assembly 300 can be different types of function-performing effectors such as hinged graspers, rotating appendages, extending appendages, or other configurations that enable rehabilitative movements and/or tasks to be performed. Furthermore, effector assembly 300 can be configured for other parts of the body such as a wrist, arm, foot, leg, ankle or shoulder. Wearable assembly 400 is illustrated with a housing 405 similar to main housing component 124 of FIGS. 1A-1B. In other embodiments, housing 405 can be shaped and sized for the particular body part that is being addressed.

System 301 includes a controller which can be an external controller 450a (e.g., a computer tablet or smartphone) or an internal controller 450b within the housing 405 of the wearable assembly. Controller 450a,b controls movements of components in the wearable assembly 400 and consequently the effector assembly 300.

A locking mechanism embodied as levers 410 secures the effector assembly 300 to the wearable assembly 400. In this embodiment, the locking mechanism is configured as a pair of levers 410 that are on opposite lateral sides of the wearable assembly 400. The levers 410 are in a closed position against the wearable assembly 400 in FIG. 3A. A user flips the levers 410 outward as shown in FIG. 3B to unlock the effector assembly 300 from the wearable assembly 400. After the levers 410 have been unlocked, the effector assembly 300 can be decoupled from the wearable assembly 400 as shown in FIG. 3C. FIG. 3D shows the effector assembly 300 being fully separated from wearable assembly 400, with a tension element 310 extending from the effector assembly 300. The tension element 310 serves as both an actuation component of effector assembly 300 as well as a coupling element between effector assembly 300 and wearable assembly 400. Tension element 310 is embodied as a linear push-pull wire in this example.

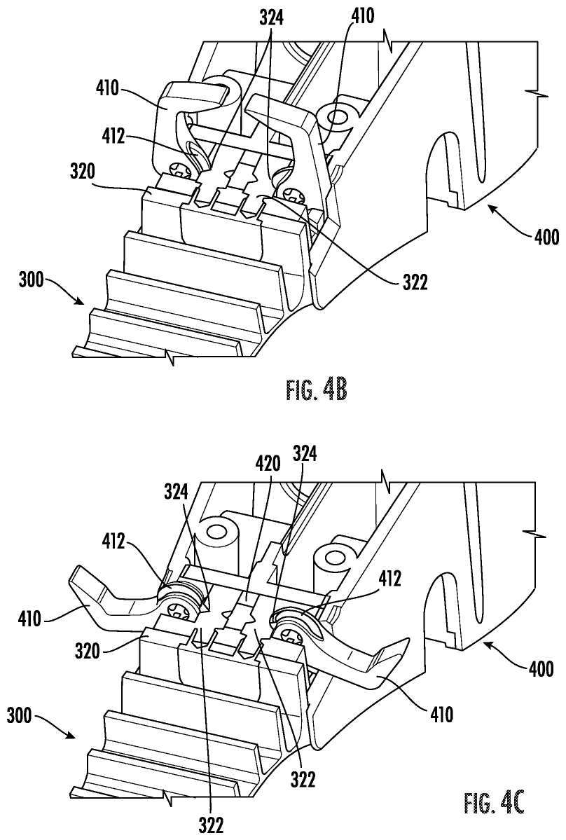

FIGS. 4A-4C show details of how the effector assembly 300 and wearable assembly 400 lock together. FIG. 4A is an isometric view facing an end surface 320 of the effector assembly 300, while FIGS. 4B and 4C are partial cross-sectional views facing toward the wearable assembly 400. The end surface 320 and end plate 420 serve as coupling interfaces between the effector assembly 300 and wearable assembly 400. The end plate 420 has the locking mechanisms (e.g., levers 410) of the wearable assembly 400 that lock onto locking features of the effector assembly 300. End surface 320 is shown as a flat plate but can be configured in other manners such as a convex or concave surface. Tension element 310 extends out of end surface 320, to be coupled to the wearable assembly 400. In the embodiment shown, locking features of the effector assembly 300 are posts 322 that protrude from the end surface 320. Posts 322 have waists 324 for the levers 410 to engage with.

FIGS. 4B and 4C show the effector assembly 300 installed on wearable assembly 400, providing a horizontal cross-section (except for levers 410) to illustrate the locking between effector assembly 300 and wearable assembly 400. The posts 322 have tapered waists 324 that are engaged by fins 412 on the inner surfaces of the levers 410. When the levers 410 are upward in a locked position as shown in FIG. 4B, the fins 412 engage the waists 324 of posts 322. When the levers 410 are hinged outward in an unlocked position as shown in FIG. 4C, the fins 412 are rotated away from the waists 324 of posts 322, thereby releasing the effector assembly 300. The locking between the effector assembly 300 and wearable assembly 400 can be operated by one hand of a user, which is beneficial for stroke patients. Other types of locking mechanisms and locking features can be used in other embodiments, such as a sliding lock or a latch on the wearable assembly that secures onto a notch or protrusion on the effector assembly.

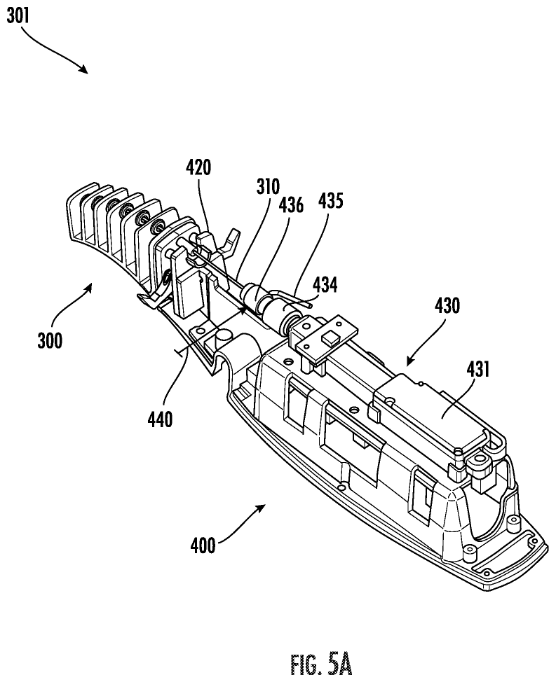

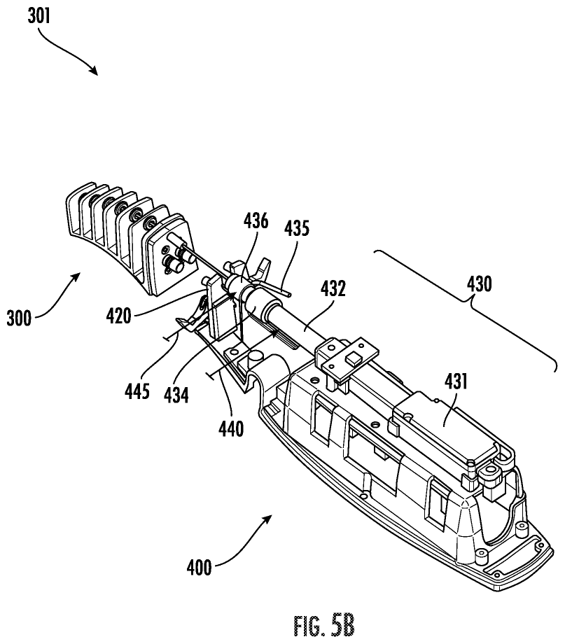

FIGS. 5A and 5B are isometric views of the orthosis system 301, where the housing 405 (FIG. 3A) has been removed to illustrate details of components inside the wearable assembly 400, in accordance with some embodiments. Wearable assembly 400 has a motor mechanism 430 that includes a motor 431 and a linear actuator 432 (FIG. 5B). Motor mechanism 430 is controlled by a controller (e.g., controllers 450a,b of FIG. 3A), controlling operation of motor 431 which moves linear actuator 432. For example, a BCI component (e.g., BCI component 215 of FIG. 2B) may be in communication with motor mechanism 430 to control movements of the motor mechanism 430. In some embodiments, motor 431 and linear actuator 432 may be supplied together as a single component. In other embodiments, motor 431 and linear actuator 432 may be configured as other types of actuation mechanisms, such as pneumatic actuators or gear assemblies.

A sensor 434 and a coupler 436 are mounted on an end of the linear actuator 432, to be moved lengthwise along the wearable assembly 400. The sensor 434 may be, for example, a load cell with wiring 435. Other types of sensors may be used such as position sensors (e.g., optical, proximity), other force sensors (e.g., strain gauge, pressure sensor), and limit switches. The coupler 436 receives and holds the tension element 310 when the effector assembly 300 is coupled to the wearable assembly 400.

Coupler 436 has a starting location 440 in which the coupler 436 is at an initial position toward the motor 431 and away from the end plate 420, as illustrated in FIG. 5A. Coupler 436 has an unloading location 445 illustrated in FIG. 5B in which the coupler 436 is at the end plate 420. In the unloading location 445, the effector assembly 300 can be unloaded (i.e., decoupled, removed) from the wearable assembly 400, to be replaced by a different effector.

During normal operation of the orthosis system 301 by a user, the effector assembly 300 is attached to the wearable assembly 400 via the levers 410 being secured to posts 322 and the tension element 310 being held within coupler 436. Instructions from the user (e.g., brain signals) or from a control system (e.g., a mode using pre-programmed rehabilitation movements) cause the motor 431 to move the linear actuator 432 forward and backward, thus actuating the effector assembly 300 by pushing and pulling the tension element 310. When the user desires to detach the effector assembly 300, the coupler 436 is moved to the unloading location 445. The unloading location 445 is further forward (i.e., toward the end plate 420) than the range of movement of the coupler 436 during normal operation. At the unloading location 445, the coupler 436 is pressed against the end plate 420, which causes the coupler 436 to release the tension element 310. The user may choose to decouple the effector assembly, for example, to replace it with another type of effector for performing different tasks or motions, or to attach a different effector than was used by a previous patient, or for ease of storing the wearable assembly without the effector assembly attached. By enabling interchangeability of effector assemblies, a single base wearable assembly can offer a variety of functionality for one patient or multiple patients. This interchangeability saves costs, improves convenience, and provides new functionality for the orthosis system.

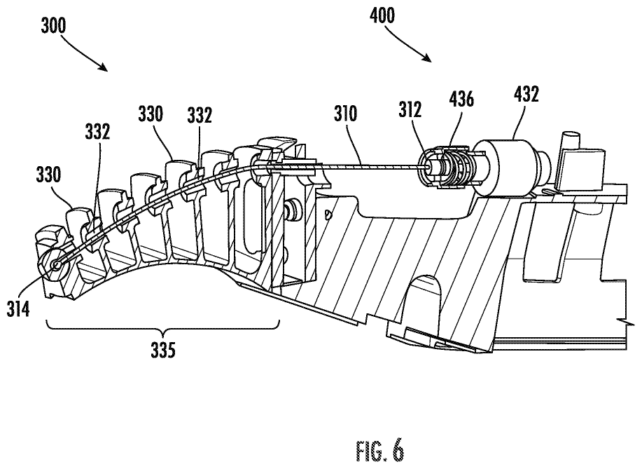

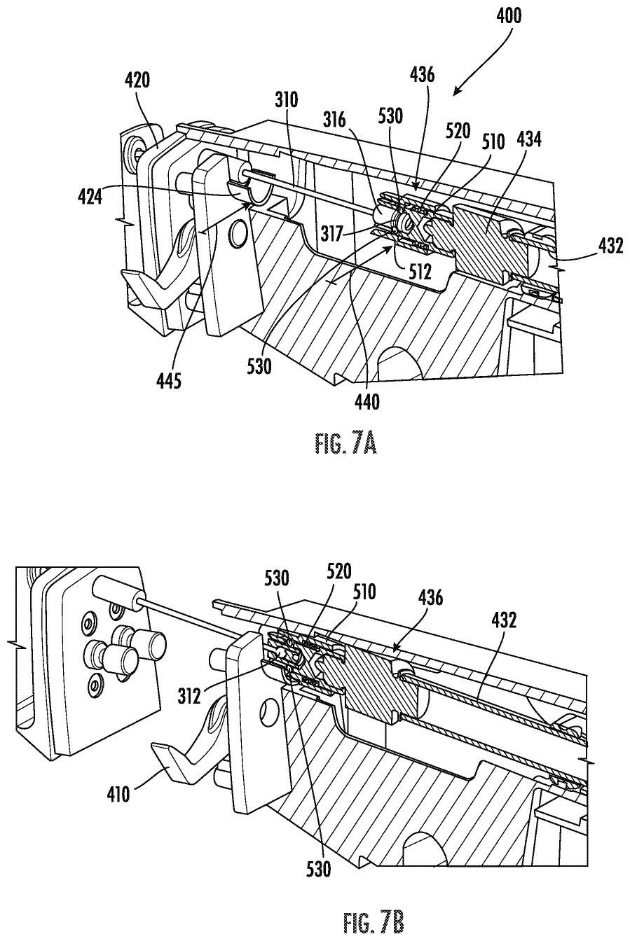

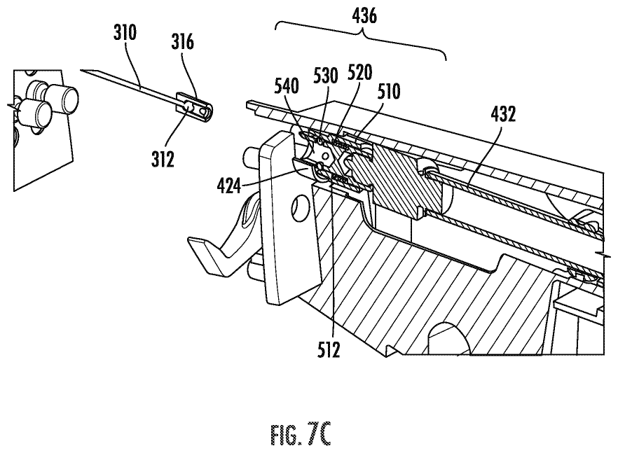

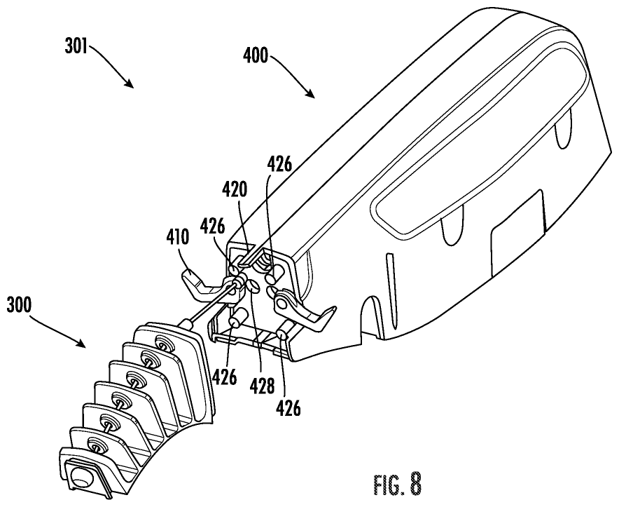

FIGS. 6 and 7A-7C show further details of the coupling mechanisms of the orthosis system that enable interchangeability of the effector assembly 300. In particular, the coupling mechanisms advantageously only require the use of one hand of the patient. FIG. 6 shows a cross-section of the effector assembly 300, depicting a plurality of baffles 330 similar to the flexible intermediate structure 128 of FIG. 1A. The baffles 330 form an effector portion 335 of the effector assembly, which is a flexible appendage in this embodiment. Effector portion 335 is the movable portion that performs the functional motions of the effector assembly 300, compared to other components that are not involved in producing motion (e.g., force sensing module 130, finger stay 122 of FIG. 1A). Baffles 330 have openings 332 within them, forming a channel through which the tension element 310 slides to actuate the effector portion 335. A first end 312 of the tension element 310 is seated in coupler 436, while a second end 314 of the tension element 310 is attached within the effector assembly 300. In this embodiment, the second end 314 is attached to a distal baffle of the effector portion 335 so that all the baffles 330 are moved by the tension element 310. When the tension element 310 is pushed and pulled by the motor of the wearable assembly, the effector assembly flexes and extends, respectively.

FIG. 7A is a partial cross-sectional view showing an initial state of the orthosis system when a user desires to uncouple the effector assembly from the orthosis device. FIG. 7B is the same view as FIG. 7A but with the coupler 436 in an unloading location that allows the effector assembly to be removed. FIG. 7C shows the tension element 310 removed from the coupler 436, thus decoupling the effector assembly from the wearable assembly.

In FIG. 7A, the user prepares the orthosis device for detaching the effector assembly by, in some embodiments, selecting an “unloading” mode (which may also be referred to as, for example, a “change” mode) on a user interface device (e.g., controller 350a of FIG. 3A) or on the orthosis device itself (e.g., push button, switch, or touch screen that connects to controller 350b of FIG. 3A in the wearable assembly). That is, the controller 350a,b has an unloading mode that moves the coupler 436 to the unloading location 445. The unloading or change mode instructs the coupler 436 to move to starting location 440 as depicted in FIG. 7A, where starting location 440 is shown relative to approximately the middle of coupler 436. In this embodiment, the starting location 440 corresponds to having the baffles in a naturally relaxed or resting state so that when the user releases the levers 410 to unlock the effector assembly 300 from the wearable assembly 400, the baffles will not be pre-loaded with any spring force. In other words, having the effector portion 335 in a resting state prevents spring-back when the effector assembly 300 is decoupled from the wearable assembly 400, thus providing case of use and safety for the user. In the embodiment shown, the natural state of the baffles 330 is in the extended finger state shown in FIG. 1B rather than a flexed finger state as shown in FIG. 1A. Accordingly, the starting location 440 moves the coupler 436 to be retracted toward the motor to situate the baffles 330 in this resting state. The natural or resting state may correspond to the shape in which the baffles (or other effector configuration that is being used) were initially manufactured (e.g., molded or machined). In other embodiments, other types of effector assemblies can require the coupler 436 to have a different starting location such as at a location partially between the starting location 440 and unloading location 445 depicted in FIG. 7A. After the unloading mode has been selected, the user unlocks the locking mechanisms, such as by flipping levers 410 outward, and removes their fingers (or other body part that is being treated) from the orthosis device (e.g., out of the finger carriage).

Coupler 436 has a sleeve 510, a spring 520 inside the sleeve 510, and an engagement element 530 which is embodied as ball bearings. The sleeve 510 is a collar in this embodiment that surrounds a receiving space 540 (shown in FIG. 7C). The first end 312 of the tension element 310 is seated in the receiving space 540 when the effector assembly is coupled to the wearable assembly. The first end 312 includes a securing feature that assists the coupler 436 in holding the tension element 310, where the securing feature is illustrated as a knob 316 in this embodiment. Knob 316 is illustrated as a cylinder with a larger diameter than the diameter of the wire of tension element 310. That is, tension element 310 has a first diameter, and knob 316 (i.e., securing feature) has a second diameter that is greater than the first diameter. Knob 316 has a waist 317 that engagement elements 530 can engage with. In other embodiments, the securing feature can be configured in other manners that enable the tension element 310 to be held by the coupler 436, such as a hook, a loop, or having ridged surface or indentations on the tension element itself. The securing feature (e.g., knob 316) can be a separate component joined to the tension element 310 or can be integrally formed with first end 312 of the tension element 310. In some embodiments, such as depicted in FIGS. 7A-7C, the knob 316 and receiving space 540 are circumferentially symmetrical (i.e., the same shape in any rotation) to enable the user to easily insert the tension element 310 into the wearable assembly 400 rather than having to align the knob 316 in a particular orientation.

The coupler 436 is configured as a quick release coupling in this embodiment. In normal operation and when the coupler 436 is not at the unloading location 445 (e.g., in FIG. 7A), the spring 520 is biased to hold sleeve 510 in an engaged position. In the engaged position, sleeve 510 is forward such that the engagement elements 530 protrude into the receiving space 540, being seated in waist 317 of knob 316 to hold the tension element 310. When the coupler 436 is at the unloading location 445 (e.g., FIG. 7B), a shoulder 512 of sleeve 510 contacts lip 424 of end plate 420. Lip 424 is aligned with sleeve 510. As the linear actuator 432 presses shoulder 512 against lip 424, sleeve 510 is slid proximally relative to the receiving space 540. The sleeve 510 is consequently moved by the lip 424 to a disengagement position that allows the engagement elements 530 to be withdrawn from the receiving space 540, thereby releasing the knob 316 (and first end 312) of the tension element 310.

Because the levers 410 are unlocked in FIG. 7B, the coupler 436 pushes the tension element 310 and the entire effector assembly 300 away from the wearable assembly 400 as the coupler 436 is moved to the unloading location 445, rather than actuating the effector portion 335 as would occur during normal operation. The user can then remove the effector assembly 300 from the wearable assembly 400 as shown in FIG. 7C, to interchange the effector with a different effector or to store it. In some embodiments, the motor can be programmed to operate at a slower speed or to decelerate as it moves during the unloading mode, to allow time for the patient to hold onto the effector assembly before the effector is released. Some embodiments may include an accessory such as a tool, fixture, or enclosing case to hold or immobilize the movable parts (e.g., baffles 330) of the effector portion 335 during removal of the effector assembly 300, to facilitate handling by the user.

In embodiments, an orthosis system has a wearable assembly and a user replaceable orthosis effector assembly. The wearable assembly includes a motor mechanism and a coupler. The coupler is movable by the motor mechanism between a starting location and an unloading location, and the coupler has a receiving space. The user replaceable orthosis effector assembly couples to the wearable assembly. The effector assembly has a tension element that actuates the effector assembly. A first end of the tension element is seated in the receiving space of the coupler when the effector assembly is coupled to the wearable assembly. The first end of the tension element is releasable from the receiving space when the coupler is in the unloading location, decoupling the effector assembly from the wearable assembly.

In some embodiments, the wearable assembly further comprises an end plate at the unloading location, the end plate having a lip that contacts the coupler when the coupler is at the unloading location. In some embodiments, the coupler further comprises a sleeve and an engagement element; the sleeve has an engaged position in which the engagement element protrudes into the receiving space to engage the first end of the tension element; and the lip moves the sleeve to a disengagement position in which the engagement element withdraws from the receiving space, releasing the first end of the tension element.

In some embodiments, the wearable assembly further comprises a sensor coupled to the coupler, and the sensor senses that the tension element is inserted into the receiving space. In some embodiments, when the sensor senses that the tension element has been inserted into the receiving space, the motor mechanism moves the coupler away from the unloading location, causing the coupler to engage the first end of the tension element. In some embodiments, the sensor is a load cell. In some embodiments, the orthosis system further includes a controller that controls the motor mechanism, wherein in an unloading mode, the controller moves the coupler to the unloading location, and wherein the controller moves the coupler away from the unloading location when the sensor senses that the first end of the tension element has been inserted into the receiving space.

In some embodiments, the tension element is a wire. In some embodiments, the first end of the tension element comprises a knob that is seated in the receiving space of the coupler when the effector assembly is coupled to the wearable assembly. In some embodiments, the effector assembly further comprises an effector portion; the tension element slides within the effector portion to actuate the effector portion; the tension element has a knob at the first end of the tension element; the first end of the tension element extends out of an end surface of the effector assembly; and a second end of the tension element is attached within the effector portion.

In some embodiments, the motor mechanism comprises a motor coupled to a linear actuator. In some embodiments, the wearable assembly further comprises a locking mechanism that locks with a locking feature on the effector assembly. In some embodiments, the orthosis system further comprises a brain-controlled interface (BCI) component that is in communication with the motor mechanism. In some embodiments, the BCI component is controlled by brain signals, muscle signals, or kinetic signals. In some embodiments, the wearable assembly is configured for an upper extremity of a human body. In some embodiments, the wearable assembly is configured for a lower extremity of a human body.

As can be understood from this disclosure, embodiments beneficially use the tension element of the effector assembly as both an actuation component of the effector assembly as well as a coupling component to join the effector assembly to the wearable assembly 400. In doing so, interchangeability of the effector is achieved with components that are already part of an orthosis system, rather than requiring more components which adds cost and complexity. The tension element serves as a universal actuator for the effectors being used with the base wearable assembly. Additionally, the tension element is beneficially configured to be decoupled or attached to the base assembly in a manner that can be performed by one hand of the user.

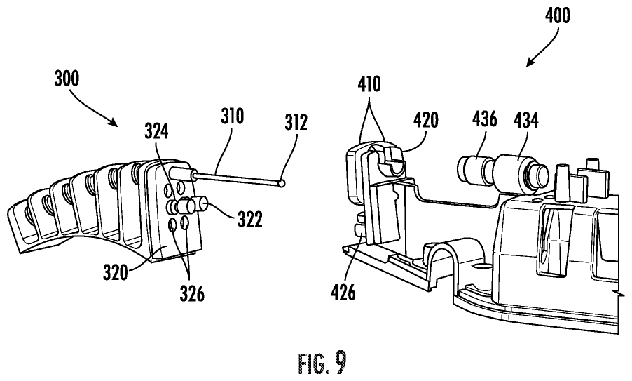

FIGS. 8 and 9 illustrate features for installing the effector assembly onto the wearable assembly. FIG. 8 is an isometric view of the orthosis system 301, facing the end plate 420 of the wearable assembly 400. FIG. 9 is a side isometric view showing end surface 320 of the effector assembly 300. In both FIGS. 8 and 9, the effector assembly 300 is separated from and ready to be placed onto wearable assembly 400.

To replace an effector assembly 300 on the wearable assembly 400, a user feeds the first end 312 of the tension element 310 into the receiving space 540 of the coupler 436 in the wearable assembly 400. FIG. 9 shows an embodiment in which the first end 312 has a securing feature that is shaped as a ball having a larger diameter than the tension element 310, without the knob 316 of previous embodiments. When loading the effector assembly 300, the coupler 436 may already be at the unloading location 445 from a previous rehabilitation session, or the coupler 436 may be instructed to move to the unloading location. For instance, a user may instruct the wearable assembly to prepare for installing an effector assembly by selecting the unloading or change mode via a user interface device (e.g., smartphone or computer tablet) or from an input interface on the wearable assembly itself. The change mode causes the motor to move the coupler 436 to the unloading location 445. As the user inserts the tension element into the wearable assembly 400, the user aligns the posts 322 with the levers 410 so that the effector assembly 300 can be locked into place. Some embodiments may include recesses 428 in end plate 420 for receiving the posts 322, to further aid the user in easily mounting the effector assembly 300 onto the wearable assembly 400.

In the embodiment of FIG. 8, the end plate 420 also includes spacers 426 which are pins that extend outwardly from the end plate 420. Four spacers 426 are shown in this embodiment, but other quantities may be used such as one to three spacers. The spacers 426 may also be configured in other shapes such as rectangular protrusions, rather than cylindrical pins as shown. The spacers 426 fit into recesses 326 of end surface 320, shown in FIG. 9. The spacers 426 help ensure that as the levers 410 engage the posts 322, the effector assembly 300 and the wearable assembly 400 will be locked together in a repeatable manner. In other words, the spacers 426 serve as stops so that the motor 431 of the wearable assembly 400 is zeroed in the same position (e.g., starting location 440) each time that effector assembly is used. In embodiments using the levers 410 as locking mechanisms, the levers 410 engage the tapered waists 324 of the posts 322, pulling the posts 322 into the recesses 428, but the spacers 426 limit how far. This consistent positioning helps provide proper actuation of the effector and enables accurate tracking of movement data. The spacers 426 enable the effector assembly to be reliably positioned with respect to a base orthosis device, rather than requiring calibration each time a different effector is attached. In some embodiments, different effectors may have different actuation movement distances of the tension element 310 (e.g., longer or shorter ranges of motion). The spacers 426 can help establish the required starting location for the motor for a particular effector assembly by positioning the effector assembly relative to the wearable assembly in a repeatable manner.

When the user inserts the tension element 310 into the receiving space 540, the wearable assembly 400 detects that an effector assembly is being installed and automatically secures the tension element 310 into the coupler 436. This automatic detection beneficially enables a one-handed replacement of the effector assembly by the user. In some embodiments, the detection involves the sensor 434 recognizing that the tension element 310 has been placed into the receiving space 540. The sensing can be, for example, a force of the tension element (e.g., tension wire) being imparted on the coupler due to the user inserting the wire. Other types of sensing may be utilized, such as optical sensors, electromagnetic sensors (e.g., proximity sensors), or mechanical limit switches identifying when the tension element is inserted. The sensor 434 is coupled to the coupler 436, such as being mounted on an external surface of the coupler 436 or within the receiving space 540, depending on the type of sensor being used.

The orthosis system 301 can be programmed to ensure that the coupler 436 is not moved prematurely, before the user is ready to attach the effector assembly. For example, in an embodiment of sensor 434 being a load cell, the orthosis system can be programmed to determine that the tension element is in place when a force above a certain threshold is detected, or that a change in force of a certain magnitude has been reached. The system can also require that the force criteria (e.g., force value or change in force) be met for a certain period of time before moving the coupler 436, such as one to three seconds. The force and/or time criteria can also help ensure that the tension wire is fully inserted into the coupler.

The controller moves the coupler 436 away from the unloading location 445 when the sensor 434 senses that the first end 312 of the tension element 310 has been inserted into the receiving space 540. That is, when the system determines that the tension element 310 is in place, using readings from sensor 434, the motor 431 moves the linear actuator 432. As a result, the coupler 436 is moved away from the unloading location 445 (e.g., moving from the location shown in FIG. 7B to that of FIG. 7A), which allows the sleeve 510 to slide back into its normally biased position. The engagement elements 530 then protrude into the receiving space 540 to engage the knob 316 and secure the tension element 310. The user closes levers 410 into the posts 322 to lock the effector assembly 300 onto the wearable assembly 400.

Other types of mechanisms can be used in coupler 436 to hold tension element 310, in other embodiments. In one example, a chuck can be used to hold the tension element 310, where jaws of the chuck can be configured as a quick release component by a spring, magnet, or other mechanism. In another example, the tension element 310 can be surrounded by an inflatable sleeve which is activated hydraulically or pneumatically. When inflated, the sleeve is designed with sufficient length (and thus surface area) to grab onto and secure the tension element 310.

In some embodiments a wearable assembly that couples to a user replaceable orthosis effector assembly includes a motor mechanism, a coupler, and an end plate. The coupler is movable by the motor mechanism between a starting location and an unloading location, wherein the coupler has a receiving space, a sleeve and an engagement element. The end plate is at the unloading location, the end plate having a lip that is aligned with the sleeve. The sleeve is biased toward an engaged position in which the engagement element protrudes into the receiving space. When the coupler is in the unloading location, the lip moves the sleeve to a disengagement position, the engagement element being withdrawn from the receiving space in the disengagement position.

In some embodiments, the sleeve is a collar around the receiving space. In some embodiments, the engagement element is a ball bearing. In some embodiments, the motor mechanism comprises a motor coupled to a linear actuator. In some embodiments, the end plate has a locking mechanism that locks onto a locking feature of the effector assembly. In some embodiments, the locking mechanism is a lever with a securing feature. In some embodiments, the wearable assembly further comprises a spacer that extends from the end plate.

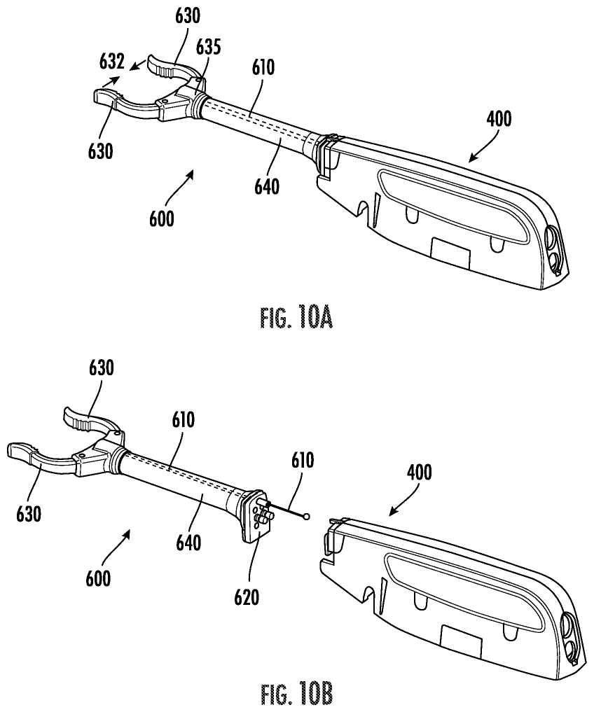

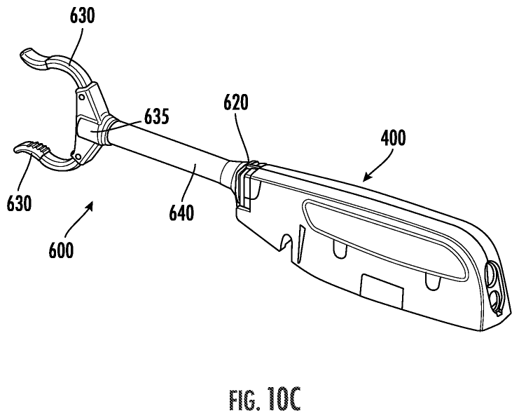

FIGS. 10A-10C show isometric views of an example of an alternative effector assembly 600 that can be interchanged with the effector assembly 300. FIG. 10A shows effector assembly 600 mounted to wearable assembly 400, and FIG. 10B shows effector assembly 600 detached from wearable assembly 400. FIG. 10C shows effector assembly 600 mounted to wearable assembly 400 but in an orientation that is rotated 90 degrees relative to wearable assembly 400 compared to the view shown in FIG. 10A. The effector 600 has opposing appendages 630 that allow a user to grasp or hold an object. The effector 600 can be utilized, for example, as an all-purpose gripper or may have the appendages 630 shaped to pick up particular objects such as a cup or toothbrush. The appendages 630 may be shaped identically to each other as shown in FIGS. 10A-10C or may be shaped differently from each other (e.g., one appendage being curved and the other one straight, or the appendages being of different length than each other). The appendages 630 may include padding or a grippable material on their surfaces to assist in holding the objects being picked up.

The appendages 630 are attached to an arm 640 through which tension element 610 runs. The tension element 610 actuates the appendages 630, such as being coupled to the appendages 630 via joint 635. In one embodiment, when the tension element 610 is pulled by wearable assembly 400, the tips of appendages 630 move together as indicated by arrows 632. FIG. 10B shows the tension element 610 extending out of end surface 620, such that the tension element 610 serves as a component for attaching effector assembly 600 to wearable assembly 400 as explained previously in this disclosure. FIG. 10C shows that the appendages can be configured to be adjustably oriented on arm 640, such as at joint 635 or at the opposite end of arm 640 near end surface 620, to facilitate a greater field of use. For example, joint 635 can be configured to enable rotation of the appendages 630 in discrete intervals (e.g., 15-degree increments) and lock in place at the desired orientation.

Other embodiments of user replaceable orthosis effector assemblies include different sizes of a particular design. For example, the finger flexing effector assembly 300 disclosed herein can be provided in different lengths and/or widths to accommodate pediatric or elderly patients. In another example, the interchangeable effectors can be designed to attach to different fingers of a patient or different numbers of fingers (e.g., index and middle fingers, or all four fingers excluding the thumb). Similarly, interchangeable lower extremity effectors can enable different leg, foot or toe sizes to be accommodated.

Further embodiments of effectors can provide different types of motion. For example, an effector assembly that flexes up and down can be replaced by an effector assembly that provides circular/conical rotational movement. Different effector assemblies can also provide different ranges of motion, such as smaller ranges for patients beginning their rehabilitation program. The motions provided by the interchangeable effector assemblies can be configured for the body part on which the orthosis system is being worn. Examples include flexion, extension, rotation, and other movements of the shoulder, elbow, wrist, fingers, hip, knee, leg, ankle, foot and toes.

In some embodiments, a user replaceable orthosis effector assembly that couples to a wearable assembly includes an effector portion, a tension element and a securing feature. The tension element moves within the effector portion to actuate the effector portion. The tension element has a first end extending out of an end surface of the effector assembly and a second end attached within the effector portion. The securing feature is at the first end of the tension element.

In some embodiments, the tension element is a wire. In some embodiments, the effector assembly further comprises a locking feature on the end surface. In some embodiments, the tension element has a first diameter, and the securing feature has a second diameter that is greater than the first diameter. In some embodiments, the effector portion comprises a flexible appendage comprising a plurality of baffles having openings, and the tension element slides through the openings in the plurality of baffles. In some embodiments, the effector portion comprises opposing appendages configured to grasp an object.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. An orthosis system comprising:
i) a wearable assembly having:
a motor mechanism; and
a coupler that is movable by the motor mechanism between a starting location and an unloading location, wherein the coupler has a receiving space; and
ii) a user replaceable orthosis effector assembly that couples to the wearable assembly, the effector assembly having a tension element that actuates the effector assembly;
wherein a first end of the tension element is seated in the receiving space of the coupler when the effector assembly is coupled to the wearable assembly; and
wherein the first end of the tension element is releasable from the receiving space when the coupler is in the unloading location, decoupling the effector assembly from the wearable assembly.

2. The system of claim 1, wherein the wearable assembly further comprises an end plate at the unloading location, the end plate having a lip that contacts the coupler when the coupler is at the unloading location.

3. The system of claim 2, wherein:
the coupler further comprises a sleeve and an engagement element;
the sleeve has an engaged position in which the engagement element protrudes into the receiving space to engage the first end of the tension element; and
the lip moves the sleeve to a disengagement position in which the engagement element withdraws from the receiving space, releasing the first end of the tension element.

4. The system of claim 1, wherein:
the wearable assembly further comprises a sensor coupled to the coupler; and
the sensor senses that the tension element is inserted into the receiving space.

5. The system of claim 4, wherein when the sensor senses that the tension element has been inserted into the receiving space, the motor mechanism moves the coupler away from the unloading location, causing the coupler to engage the first end of the tension element.

6. The system of claim 4, wherein the sensor is a load cell.

7. The system of claim 4, further comprising a controller that controls the motor mechanism;
wherein in an unloading mode, the controller moves the coupler to the unloading location; and
wherein the controller moves the coupler away from the unloading location when the sensor senses that the first end of the tension element has been inserted into the receiving space.

8. The system of claim 1, wherein the tension element is a wire.

9. The system of claim 1, wherein the first end of the tension element comprises a knob that is seated in the receiving space of the coupler when the effector assembly is coupled to the wearable assembly.

10. The system of claim 1, wherein:
the effector assembly further comprises an effector portion;
the tension element slides within the effector portion to actuate the effector portion;
the tension element has a knob at the first end of the tension element;
the first end of the tension element extends out of an end surface of the effector assembly; and
a second end of the tension element is attached within the effector portion.

11. The system of claim 1, wherein the motor mechanism comprises a motor coupled to a linear actuator.

12. The system of claim 1, wherein the wearable assembly further comprises a locking mechanism that locks with a locking feature on the effector assembly.

13. The system of claim 1, further comprising a brain-controlled interface (BCI) component that is in communication with the motor mechanism.

14. The system of claim 13, wherein the BCI component is controlled by brain signals, muscle signals, or kinetic signals.

15. The system of claim 1, wherein the wearable assembly is configured for an upper extremity of a human body.

16. The system of claim 1, wherein the wearable assembly is configured for a lower extremity of a human body.

17. A user replaceable orthosis effector assembly that couples to a wearable assembly, the effector assembly comprising:
an effector portion;
a tension element that moves within the effector portion to actuate the effector portion, the tension element having a first end extending out of an end surface of the effector assembly and a second end attached within the effector portion; and
a securing feature at the first end.

18. The effector assembly of claim 17, wherein the tension element is a wire.

19. The effector assembly of claim 17, further comprising a locking feature on the end surface.

20. The effector assembly of claim 17, wherein:
the tension element has a first diameter; and
the securing feature has a second diameter that is greater than the first diameter.

21. The effector assembly of claim 17, wherein:
the effector portion comprises a flexible appendage comprising a plurality of baffles having openings; and
the tension element slides through the openings in the plurality of baffles.

22. The effector assembly of claim 17, wherein the effector portion comprises opposing appendages configured to grasp an object.

23. A wearable assembly that couples to a user replaceable orthosis effector assembly, the wearable assembly comprising:
a motor mechanism;
a coupler movable by the motor mechanism between a starting location and an unloading location, wherein the coupler has a receiving space, a sleeve and an engagement element; and
an end plate at the unloading location, the end plate having a lip that is aligned with the sleeve;
wherein the sleeve is biased toward an engaged position in which the engagement element protrudes into the receiving space; and
wherein when the coupler is in the unloading location, the lip moves the sleeve to a disengagement position, the engagement element being withdrawn from the receiving space in the disengagement position.

24. The wearable assembly of claim 23, wherein the sleeve is a collar around the receiving space.

25. The wearable assembly of claim 23, wherein the engagement element is a ball bearing.

26. The wearable assembly of claim 23, wherein the motor mechanism comprises a motor coupled to a linear actuator.

27. The wearable assembly of claim 23, wherein the end plate has a locking mechanism that locks onto a locking feature of the effector assembly.

28. The wearable assembly of claim 27, wherein the locking mechanism is a lever with a securing feature.

29. The wearable assembly of claim 23, further comprising a spacer that extends from the end plate.
